(19) United States
(12) Patent Application Publication
(10) Pub. No.: US 2020/0399197 A1
(43) Pub. Date: Dec. 24, 2020

(54) Glycol Ether Ester Compounds Useful In Lubricating Oil Compositions And Methods Of Making Same

(71) Applicant: ExxonMobil Research and Engineering Company, Annandale, NJ (US)

(72) Inventors: Abhimanyu O. Patil, Westfield, NJ (US); Satish Bodige, Wayne, NJ (US); Kyle G. Lewis, Houston, TX (US)

(21) Appl. No.: 16/872,973

(22) Filed: May 12, 2020

Related U.S. Application Data
(60) Provisional application No. 62/864,660, filed on Jun. 21, 2019.

(51) Int. Cl. C07C 59/125; C07C 67/08; C09K 5/20; C10M 105/38

(52) U.S. Cl. CPC C07C 59/125; C07C 67/08; C09K 5/20; C10N 2030/02; C10M 2207/2835; C10M 2203/003; C10M 105/38

(57) ABSTRACT

Compositions include ether ester compounds formed from gamma-branched aliphatic alcohols, lubricating oil base stocks containing such ester compounds, and lubricating oil compositions containing such ester compounds. Methods can include making and formulating compositions containing ether ester compounds formed from gamma-branched aliphatic alcohols.

### FIELD

This disclosure relates to esters compounds, lubricating oil base stocks, and lubricating oil compositions. In particular, this disclosure relates to glycol ether ester compounds of gamma-branched alcohols, and methods of making the glycol ether ester compounds.

### BACKGROUND

Lubricants in commercial use today are often prepared from a variety of natural and synthetic base stocks admixed with various additive packages and solvents depending upon their intended application. Base stocks often include mineral oils, polyalphaolefins, gas-to-liquid base oils, silicone oils, phosphate esters, diesters, polyol esters, and the like.

The trend for passenger car engine oils (“PCEO”) is an overall improvement in quality as higher quality base stocks become more readily available. Typically, the highest quality PCEO product is formulated with base stocks such as polyalphaolefins (“PAOs”) or gas-to-liquid (“GTL”) stocks admixed with various additive packages. Polyalphaolefins are important lube base stocks with many excellent lubricant properties, including high viscosity index (“VI”) and low volatility, and are available in various viscosity range (e.g., kinematic viscosity at 100° C. in the range of 2 to 300 cSt).

However, PAOs are paraffinic hydrocarbons with low polarity. This low polarity leads to low solubility and dispersancy for polar additives or sludge generated during service. To compensate for this low polarity, lube formulators usually add one or multiple polar co-base stocks. Ester or alkylated naphthalene (AN) is usually present at 1 wt. % to 50 wt. % levels in many finished lubricant formulations to increase the fluid polarity which improves the solubility of polar additives and sludge. There is a need to have oxygen containing polar but stable Group V base stocks.

In addition, while low viscosity base stocks (kinematic viscosity at 100° C., <4 cSt) can be used to formulate next-generation ultra-low viscosity engine oils (i.e., 0W-16, 0W-12, 0W-8, “0W-4”), these base stocks require polarity to solubilize commonly used additives in lubricant formulations (PVL, CVL, industrial lubricants).

Furthermore, in order to provide step-out fuel economy while maintaining or improving other lubricant performance features, base stocks with lower friction coefficients are needed. Low friction coefficients and low viscosity at all temperature ranges are two important properties contributing to lubricant fuel economy.

Improving heat transfer is also an emerging need as the energy density of systems and equipment increases, where improving thermodynamic efficiency is often coupled with higher operating temperatures. There are also developing requirements to provide cooling fluids for hybrid and electric vehicles. Currently traditional cooling fluids, including formulated lubricants, can be used but have limited properties.

Therefore, a need exists for base stock fluids having superior viscosity, volatility characteristics and heat transfer fluid properties to meet growing industrial requirements of energy density, specific heat capacity, and thermal conductivity, while also exhibiting appropriate solubility and dispersibility for polar additives and/or sludge generated during service of lubricating oils.

### SUMMARY

Presented herein are esters of gamma-branched aliphatic alcohols (especially aliphatic alcohols) which can be advantageously used as lubricating oil base stocks with desirable lubricating oil properties such as viscosity, volatility and polarity.

Provided herein are compounds of structural Formula F-I as follows:

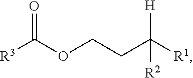

wherein R¹ and R² are independently each a C2 to C60 linear or branched alkyl group; and R³ is a glycol ether or polyglycol ether.

Further provided are lubricating oil compositions comprising an ether ester compound of Formula F-I.

Also provided are methods for making compounds of Formula F-I or lubricating oil base stocks including comprising a compound of Formula F-I as follows:

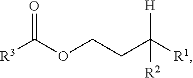

wherein R¹ and R² are independently each a C2 to C60 linear or branched alkyl group; and R³ is a glycol ether or polyglycol ether;
the method comprising the steps of:
reacting an acid of Formula F-TT as follows:

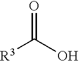

or an anhydride of Formula F-III as follows:

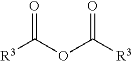

with an alcohol of Formula IV as follows:

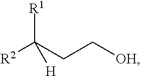

in the presence of an acid catalyst to obtain a reaction mixture; and obtaining at least a portion of the compound or the lubricating oil base stock from the reaction mixture.

Further objects, features and advantages of the present disclosure will be understood by reference to the following drawings and detailed description.

### DETAILED DESCRIPTION

### Definitions

In the present disclosure, the indefinite article “a” or “an” means at least one, unless it is clearly specified or indicated by the context to mean one.

“Alkyl group” refers to a saturated hydrocarbyl group including carbon and hydrogen atoms. “Linear alkyl group” refers to a non-cyclic alkyl group in which all carbon atoms are covalently connected to no more than two carbon atoms. “Branched alkyl group” refers to a non-cyclic alkyl group in which at least one carbon atom is covalently connected to more than two carbon atoms. “Cycloalkyl group” refers to an alkyl group in which all carbon atoms form a ring structure.

“Alkoxy group” refers to a saturated carbon hydrogen chain group singularly bonded to oxygen.

“Aryl group” refers to an unsaturated, cyclic hydrocarbyl group including carbon and hydrogen atoms in which the carbon atoms join to form a conjugated 7 system. Non-limiting examples of aryl groups include phenyl, 1-naphthyl, 2-naphthyl, 3-naphthyl, and the like.

“Arylalkyl group” refers to an alkyl group substituted by an aryl group or alkylaryl group. None-limiting examples of arylalkyl group include benzyl, 2-phenylpropyl, 4-phenylbutyl, 3-(3-methylphenyl)propyl, 3-(p-tolyl)propyl, and the like.

“Alkylaryl group” refers to an aryl group substituted by an alkyl group. Non-limiting examples of alkylaryl group include 2-methylphenyl, 3-methylphenyl, 4-methylphenyl, 2-methyl-1-naphtyl, 6-phenylhexyl, 5-pentylphenyl, 4-butylphenyl, 4-tert-butylphenyl, 4-octylphenyl, and the like.

“Cycloalkylalkyl group” refers to an alkyl group substituted by a cycloalkyl group or an alkylcycloalkyl group. An example of cycloalkylalkyl group is cyclohexylmethyl.

“Alkylcycloalkyl group” refers to a cycloalkyl group substituted by an alkyl group. Non-limiting examples of alkylcycloalkyl group include 2-methylcyclohexyl, 3-methylcyclohexyl, 4-methylcyclohexyl, 4-tert-butyl cyclohexyl, 4-phenylcyclohexyl, and the like.

“Glycol ether” refers to an ether group having a structure corresponding to the following formula:

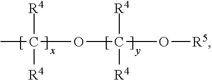

where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30) alkoxy group (C1-C30), aryl group (C4-C30), or arylalkyl group (C5-C30), x is a value from about 0 to about 10, and y is a value from about 1 to about 10.

“Polyglycol ether” refers to an ether group having a structure corresponding to the following formula:

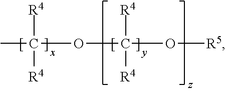

where each R4 is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30) x is a value from about 0 to about 10, y is a value from about to about 10, and z is a value from about 0 to about 100.

“Hydrocarbyl group” refers to a group including hydrogen and carbon atoms only. A hydrocarbyl group can be saturated or unsaturated, linear or branched, cyclic or acyclic, containing a cyclic structure or free of cyclic structure, and aromatic or non-aromatic.

“Cn” group or compound refers to a group or a compound including carbon atoms at total number thereof of n. Thus, “Cm-Cn” or “Cm to Cn” group or compound refers to a group or compound including carbon atoms at a total number thereof in the range from m to n. Thus, a C1-C50 alkyl group refers to an alkyl group containing carbon atoms at a total number thereof in the range from 1 to 50.

“Mono-ester” refers to a compound having one ester (—C(O)—O—) functional group therein.

“Guerbet alcohol” refers to beta-substituted alcohol having a structure corresponding to the following formula:

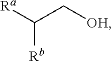

where R^{a} and R^{b} are independently linear, branched, cyclic, substituted or unsubstituted hydrocarbyl groups, which may include from c1 to c2 carbon atoms, where c1 and c2 can be, independently, 1, 2, 3, 4, 5, 6, 7, 8, 9, 10, 15, 20, 25, 30, 35, 40, 45, 50, 55, 60, 65, 70, 75, 80, 85, 90, 95, 100, as long as c1<c2. In an aspect, c1=2 and c2=50. In an aspect, R^{a} and R^{b} are alkyl groups. In another aspect, R^{a} and R^{b} are linear or branched alkyl groups.

“Gamma-branched alcohol’ refers to an alcohol having a structure corresponding to the following formula:

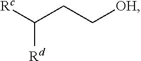

where R^{c} and R^{d} are independently linear, branched, cyclic, substituted or unsubstituted hydrocarbyl groups including from d1 to d2 carbon atoms, where d1 and d2 can be, independently, 1, 2, 3, 4, 5, 6, 7, 8, 9, 10, 15, 20, 25, 30, 35, 40, 45, 50, 55, 60, 65, 70, 75, 80, 85, 90, 95, 100, as long as d1<d2. In an aspect, d1=2 and d2=50. In an aspect, R^{c} and R^{d} are alkyl groups. In another aspect, R^{c} and R^{d} are linear or branched alkyl groups. In yet another aspect, R^{c} and R^{d} may differ in terms of total number of carbon atoms contained therein by two (2).

“SAE” refers to SAE International, formerly known as Society of Automotive Engineers, which is a professional organization that sets standards for internal combustion engine lubricating oils.

“SAE J300” refers to the viscosity grade classification system of engine lubricating oils established by SAE, which defines the limits of the classifications in rheological terms only.

“Lubricating oil” refers to a substance that can be introduced between two or more surfaces and lowers the level of friction between two adjacent surfaces moving relative to each other. A lubricating oil “base stock” is a material, typically a fluid at various levels of viscosity at the operating temperature of the lubricating oil, used to formulate a lubricating oil by admixing with other components. Non-limiting examples of base stocks suitable in lubricating oils include API Group I, Group II, Group III, Group IV, and Group V base stocks. If one base stock is designated as a primary base stock in the lubricating oil, any additional base stock may be called a co-base stock.

All kinematic viscosity values in the present disclosure are as determined pursuant to ASTM D445. Kinematic viscosity at 100° C. is reported herein as KV100, and kinematic viscosity at 40° C. is reported herein as KV40. Unit of all KV100 and KV40 values herein is cSt unless otherwise specified.

All viscosity index (“VI”) values in the present disclosure are as determined pursuant to ASTM D2270.

All Noack volatility (“NV”) values in the present disclosure are as determined pursuant to ASTM D5800 unless specified otherwise. Unit of all NV values is wt %, unless otherwise specified.

All percentages in describing chemical compositions herein are by weight unless specified otherwise. “wt %” means percent by weight.

“Consisting essentially of” means containing at a concentration by weight of at least 90 wt %, based on the total weight of the mixture in question. Thus, a lubricating oil base stock consisting essentially of a given ester compound includes that ester compound at a concentration by weight of at least 90 wt %, based on the total weight of the lubricating oil base stock.

All numerical values within the detailed description and the claims herein are modified by “about” or “approximately” the indicated value, taking into account experimental error and variations that would be expected by a person having ordinary skill in the art.

Synthesis methods of novel aliphatic monoester fluids based on selective γ-branched (‘Guerbet-Like’) C21-alcohol and glycol ethers as synthetic base stocks are provided herein. The present novel aliphatic monoesters include a series of novel esters based on selective γ-branched (‘Guerbet-Like’) C21-alcohols. As described herein, selective γ-branched (‘Guerbet-Like’) C21-alcohols were prepared by hydroformylation chemistry using umPAO C20-dimer. The selective γ-branched (‘Guerbet-Like’) C21-alcohol based glycol ether esters were then synthesized and characterized.

Among hydrocarbon fluids, metallocene catalyst-based, low viscosity fluids can have superior viscosity-volatility characteristics (i.e., mPAO3.4). The present fluids have a similar structure as these low viscosity fluids, but contain an ester and glycol ether segment (polarity, low traction) in one of the arms. Further, the present glycol ether-based fluids have similar molecular weight as mPAO3.4 (MW 428 for the ester molecule versus 420 for mPAO3.4). Yet, the viscosity of the present glycol ether esters is lower and viscosity index is higher.

The ester-based fluids of the present disclosure are polar fluids and generally have a low traction/friction coefficient. Moreover, the manufacturing process for these fluids can utilize existing equipment and follow current practice used by the ester synthetic base stock industry.

In an aspect, the lubricating oil formulations of the present disclosure can include gamma-branched alcohol-derived esters having ether functionality that modifies heat transfer properties that include density, specific heat, thermal conductivity, and the like.

### Gamma-Branched Alcohol-Derived Ester Compounds

One aspect of the present disclosure is a novel category of compounds of the structural Formula F-I as follows:

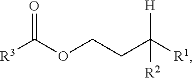

wherein R¹ and R² are independently each a hydrocarbyl group containing at least 2 carbon atoms therein (preferably a C2 to C60 hydrocarbyl group, more preferably a C2 to C60 alkyl group, still more preferably a C2 to C60 linear or branched alkyl group, still more preferably a C2 to C30 linear or branched alkyl group); and R³ is a glycol ether or polyglycol ether group. To the extent this compound can be considered as an ester derived from a gamma-branched alcohol, it will be referred to as such in the present disclosure, and it is also referred to as “ester of the present disclosure” herein.

In an aspect, R¹ and R² each independently include c1 to c2 carbon atoms, where c1 and c2 can be, independently, any integer from 2 to 60, such as 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, or 60, as long as c1<c2. In an aspect, c=2 and c2=30. In another aspect, c1=2 and c2=24. In another aspect, c1=4, and c2=16. In yet another aspect, c1=4, and c2=12. In an aspect, R¹ and R² each independently include even number of carbon atoms.

At least one of R¹ and R² (or both R¹ and R² independently each) can be a branched alkyl group, preferably a branched alkyl group of the structural Formula F-V as follows:

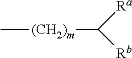

where R^{a} and R^{b} are independently hydrocarbyl groups, including alkyl groups, such as linear or branched alkyl groups. In an aspect, R^{a} and R^{b} are linear alkyl groups, m is a non-negative integer, including a non-negative integer satisfying an inequality selected from any one of m≥2, m≥3, m≥4, m≥5, m≥6, and m≥7. In an aspect, R^{a} and R^{b} each independently include c3 to c4 carbon atoms, where c3 and c4 can be, independently, any integer from 1 to 57, such as 1, 2, 3, 4, 5, 6, 7, 8, 9, 10, 12, 14, 16, 18, 20, 22, 23, 24, 26, 28, 30, 32, 34, 36, 38, 40, 42, 44, 46, 48, 50, 52, 54, 56, or 57, as long as c3<c4. In an aspect, c3 and c4 can be selected from any one of the following integer pairs: c3=1 and c4=50, c3=1 and c4=40, c3=1 and c4=20, c3=1 and c4=16, and c3=1 and c4=10. In an aspect, m=0 and R¹ and/or R² can be a group branched at the 1-location, i.e., the carbon directly connected to the quaternary carbon atom. Non-limiting examples of branched alkyls for R¹ and R² include: 2-ethylhexyl, 2-propylheptanyl, 2-butyloctyl, and 3,5-dimethyloctyl.

At least one of R¹ and R² (or both R¹ and R² independently) can be linear alkyl groups such as: ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-icosyl, n-henicosyl, n-docosyl, n-tricosyl, n-tetracosyl, n-pentacosyl, n-hexacosyl, n-octacosyl, and n-triacontyl. In an aspect, the total number of carbon atoms in linear R¹ and R² is an even number. In an aspect, the total number of carbon atoms in the linear R¹ and/or R² combined is from a1 to a2, where a1 and a2 can be, independently, 8, 10, 12, 14, 16, 18, 20, 22, 24, 26, 28, 30, 32, 34, 36, 38, 40, 42, 44, 46, 48, 52, 56, 60, 64, 80, 96, or 100, as long as a1<a2. In an aspect, the total number of carbon atoms in the linear R¹ and R² combined is within a range selected from any one of 8 to 96, 8 to 80, 8 to 64, 8 to 48, 8 to 40, 8 to 32, 8 to 28, 8 to 26, 8 to 24, 8 to 22, and 8 to 20.

In an aspect, the total number of carbon atoms in R¹ and R² combined is from b1 to b2, where b1 and b2 can be, independently, 8, 10, 12, 14, 16, 18, 20, 22, 24, 26, 28, 30, 32, 34, 36, 38, 40, 42, 44, 46, 48, 52, 56, 60, 64, 80, 96, or 100, as long as b1<b2. In an aspect, the total number of carbon atoms in R¹ and R² is in a range selected from any one of 8 to 96, 8 to 80, 8 to 64, 8 to 48, 8 to 40, 8 to 32, 8 to 28, 8 to 26, 8 to 24, 8 to 22, and 8 to 20.

In an aspect, the difference in carbon numbers contained R¹ and R² is two (2). In such case, both R¹ and R² can contain even number of carbon atoms. Thus, one of R¹ and R² can include 4, 6, 8, 10, 12, 14, 16, 18, 20, 22, 24, 26, 28, 30, 32, 34, 36, 38, 40, 42, 44, 46, 48, 50, 52, 54, 56, or 58 carbon atoms, and the other contains two more carbon atoms. In another aspect, one of R¹ and R² can include 6, 8, 10, 12, 14, 16, 18, 20, 22, 24, 26, or 28 carbon atoms, and the other group contains two more carbon atoms. In an aspect, R² can be identical to R¹—CH₂—CH₂—.

In an aspect, R³ can be a glycol ether having a structure corresponding to the following formula:

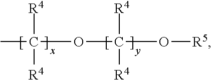

where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30) or arylalkyl group (C5-C30), x is a value from about 0 to about 10, and y is a value from about 1 to about 10.

In an aspect, R³ can be a polyglycol ether having a structure corresponding to the following formula:

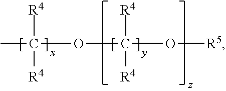

where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30) x is a value from about 0 to about 10, y is a value from about 1 to about 10, and z is a value from about 0 to about 100.

In an aspect, R³ can include up to 60, 50, 40, 30, or 20 carbon atoms. In an aspect, R³ is a C1-C24 group including carbon atoms at a number in the range from c1 to c2, where c1 and c2 can be, independently, 1, 2, 3, 4, 5, 6, 7, 8, 9, 10, 11, 12, 13, 14, 15, 16, 17, 18, 19, 20, 21, 22, 23, or 24, as long as c1<c2.

Example of ester compounds of the present disclosure include 3-octyltridecyl 2-(2-methoxyethoxy)acetate, 3-octyltridecyl 2-(3-methoxypropoxy)acetate, 3-octyltridecyl 2-(2-(2-methoxyethoxy)ethoxy)acetate, 3-octyltridecyl 2-(3-(3-methoxypropoxy)propoxy)acetate, 3-octyltridecyl 2-(2-(3-methoxypropoxy)ethoxy)acetate, 3-octyltridecyl 2-(3-(2-methoxyethoxy)propoxy)acetate, 3-decyltridecyl 2-(2-methoxyethoxy)acetate, 3-decyltridecyl 2-(3-methoxypropoxy)acetate, 3-decyltridecyl 2-(2-(2-methoxyethoxy)ethoxy)acetate, 3-decyltridecyl 2-(3-(3-methoxypropoxy)propoxy)acetate, 3-decyltridecyl 2-(2-(3-methoxypropoxy)ethoxy)acetate, 3-decyltridecyl 2-(3-(2-methoxyethoxy)propoxy)acetate, and the like.

### The Lubricating Oil Composition Containing Esters of the Present Disclosure General

The esters of the present disclosure are useful as base stocks in formulating lubricating oils. The oil composition of the present disclosure summarized above can be a portion or the entirety of a lubricating oil formulation product. Thus, the oil composition can be: (i) a base stock; (ii) a mixture of a first base stock and the remainder of the formulation absent the first base stock; (ii) a mixture of a first base stock with one or more other base stocks contained in the lubricating oil formulation absent the additive components in the lubricating oil formulation; (iii) a mixture of a first base stock and all other base stocks contained in the lubricating oil formulation but absent any additive components that may be present in the lubricating oil formulation; (iv) a mixture of the first base stock and one or more other base stocks, but not all the other base stocks, contained in the lubricating oil formulation, and at least a portion of the additive components contained in the lubricating oil formulation; and (v) a mixture of the first base stock and all additive components contained in the lubricating oil formulation, but no other base stocks contained in the lubricating oil formulation.

### Lubricating Oil Base Stocks Containing Gamma-Branched Alcohol-Derived Ester

The esters of gamma-branched alcohols of the present disclosure have desirable properties such as KV100, KV40, and viscosity index comparable to certain commercial Group V ester-type base stocks. The high polarity of the gamma-branched alcohol-derived ester molecules as a result of the presence of the ester group lends them excellent blending capabilities with many other base stocks, providing needed solvency and dispersancy of polar components such as additives and sludge formed during the service life of the lubricating oil.

The lubricating oil base stock of the present disclosure can include a single gamma-branched alcohol-derived ester compound as disclosed above at any concentration, including, e.g., up to a percent by weight of base stock (wt %) selected from any one of 80 wt %, 90 wt %, 95 wt %, 98 wt %, and 99 wt %.

In an aspect, the lubricating oil base stock of the present disclosure can include two or more gamma-branched alcohol-derived esters as disclosed above. Such base stock can be produced by mixing two ester compounds in their substantially pure form, or produced from a single esterification reaction operation by reacting (i) one or more acid(s) with two or more gamma-branched alcohols, or (ii) two or more acids with one or more gamma-branched alcohols. Such mixed-ester base stock can be particularly advantageous where a mixture of gamma-branched alcohols can be procured at a lower cost than a pure single-compound gamma-branched alcohol.

The lubricating oil base stock of the present disclosure desirably has a KV100 in the range from k1 to k2 cSt, where k1 and k2 can be, independently, 1.0, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, as long as k1<k2. In an aspect, k1=4.0, and k2=30.0. In another aspect, k1=5.0, and k2=25.0. Therefore, the base stock of the present disclosure has a relatively “low” viscosity at the normal operating temperature of an internal combustion engine lubricating oil.

The lubricating oil base stock of the present disclosure may have a viscosity index as determined pursuant to ASTM D2270 in the range from v1 to v2, where v1 and v2 can be, independently, −100, −90, −80, −70, −60, −50, −40, −30, −20, −10, 0, 10, 20, 30, 40, 50, 60, 70, 80, 90, 100, 110, 120, 130, 140, 150, 160, 170, 180, 190, 200, 210, 220, 230, 240, 250, 260, 270, 290, or 300, as long as v1<v2. In an aspect, v1 and v2 can be a pair of values selected from any one of v1=0 and v2=250, v1=25 and v2=200, and v1=100 and v2=170.

The base stock of the present disclosure may have a NV value in the range from n1 to n2 wt %, where n1 and n2 can be, independently, 0, 0.1, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90, as long as n1<n2. In an aspect, n and n2 can be a pair of values selected from any one of n1=0 and n2=50, n1=0 and n2=30, n1=0 and n2=20, and n1=0 and n2=16. In general, for the same type of gamma-branched alcohol-derived ester base stock, the larger the molecular weight of the molecule, the lower the NV value. For engine oils and base stocks containing a gamma-branched alcohol-derived ester, NV values can be lower than comparative engine oil and base stock formulations.

In an aspect, a base stock formulated in accordance with the present disclosure may have an aniline value as determined by ASTM D611 of no higher than 30, 25, 20, or 15.

Guerbet alcohol-derived esters in accordance with the present disclosure can be formulated as lubricating oil base stocks. Surprisingly, base stocks of the present disclosure that include gamma-branched alcohol-derived esters perform better than Guerbet alcohol-derived ester base stocks having similar molecular weight and with comparable molecular structure. In particular, it has been found that the gamma-branched alcohol-derived esterase stocks of the present disclosure tend to have lower viscosity (KV100, in particular), lower polarity, and/or lower volatility (NV value, in particular) than comparative PAO base stocks.

Gamma-branched alcohol-derived ester base stocks of the present disclosure can be used as a primary base stock or a co-base stock in any lubricating oil formulation. In an aspect gamma-branched alcohol-derived ester base stocks of the present disclosure can be used as a co-base stock in conjunction with a second base stock designated as a primary base stock. In certain applications, it may be desirable to include two or even more additional base stocks in the lubricating oil formulation, in addition to the gamma-branched alcohol-derived ester base stock of the present disclosure. For the convenience of description, the gamma-branched alcohol-derived ester base stock is merely referred to as a generic base stock herein, regardless of its primary base stock or co-base stock designation. The base stock of the present disclosure containing a gamma-alcohol-derived ester can be particularly advantageous when used as a co-base stock with a non-polar base stock such as those Group I, II, III, GTL, and Group IV base stocks.

The gamma-branched alcohol-derived ester base stocks of the present disclosure can be used for formulating automobile engine lubricating oils, including those meeting the SAE J300 classification standards. However, it is contemplated that the base stocks of the present disclosure may also be used to formulate other lubricating oils (e.g., automobile drive-line oils, industrial lubricating oils, gear oils, greases, and the like), heat transfer oils (e.g., transformer oils), hydraulic power transfer oils, processing oils, and the like.

### Method for Making the Ester Compounds and Lubricating Oil Base Stock Containing the Same

One aspect of the present disclosure relates to a method for making (i) a compound of Formula F-I, and/or (ii) a lubricating oil base stock comprising a compound of Formula F-I as follows:

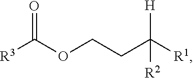

wherein R¹ and R² are independently each a C2 to C30 linear or branched alkyl group and
R³ is a hydrocarbyl group,
the method comprising the steps of:
reacting an acid of Formula F-II (below) or an anhydride thereof of Formula F-III (below) with an alcohol of Formula F-IV (below) in the presence of an acid catalyst to obtain a reaction mixture:

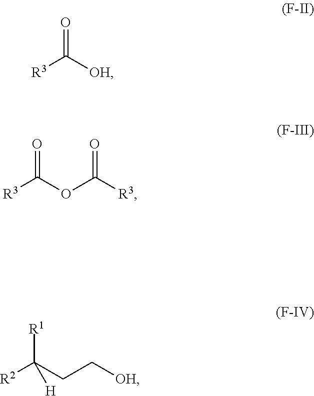

and obtaining the compound from the reaction mixture.

It is highly desirable that the acid/anhydride used in the reaction are those of a single mono-acid for both the purpose of making a single compound of Formula F-I or a lubricating oil base stock containing a compound of Formula F-I, although those of multiple acids can be used as well, especially for the purpose of making a lubricating oil base stock which can include a mixture of multiple, different compounds having a molecular structural Formula F-I.

As noted above, in the acid or anhydride, R³ can be a glycol ether having a structure corresponding to the following formula:

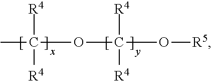

where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30), x is a value from about 0 to about 10, and y is a value from about 1 to about 10.

In an aspect, R³ can be a polyglycol ether having a structure corresponding to the following formula:

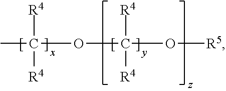

where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30), x is a value from about 0 to about 10, y is a value from about 1 to about 10, and z is a value from about 0 to about 100.

Illustrative compounds of the structural Formula F-I include glycol ether acids such as methoxyacetic acid, methoxypropionic acid, methoxyethoxyacetic acid, methoxyethoxyethoxyacetic acid, ethoxyacetic acid, ethoxyethoxyacetic acid, ethoxyethoxyethoxyacetic acid, propoxyacetic acid, propoxyethoxyacetic acid, propoxyethoxyethoxyacetic acid, butoxyacetic acid, butoxyethoxyacetic acid, butoxyethoxyethoxyacetic acid, propoxybenzoic acid, and the like.

Illustrative compounds of the structural Formula F-II can also include compounds prepared from a reaction of functionalized carboxylic acid, such as a hydroxy acid or haloacid, and a glycol ether alcohol, where the glycol ether alcohol may include, for example, methoxyethanol, ethoxyethanol, propoxyethanol, butoxyethanol, pentoxyethanol, hexyloxyethanol, phenoxyethanol, diethylene glycol methyl ether, diethylene glycol ethyl ether, diethylene glycol propyl ether, diethylene glycol butyl ether, diethylene glycol pentyl ether, diethylene glycol hexyl ether, diethylene glycol benzyl ether, triethylene glycol methyl ether, triethylene glycol ethyl ether, triethylene glycol propyl ether, triethylene glycol butyl ether, triethylene glycol pentyl ether, triethylene glycol hexyl ether, triethylene glycol benzyl ether, tetraethylene glycol methyl ether, tetraethylene glycol ethyl ether, tetraethylene glycol propyl ether, tetraethylene glycol butyl ether, tetraethylene glycol pentyl ether, tetraethylene glycol hexyl ether, tetraethylene glycol benzyl ether, propylene glycol methyl ether, propylene glycol ethyl ether, propylene glycol propyl ether, propylene glycol butyl ether, propylene glycol hexyl ether, propylene glycol benzyl ether, dipropylene glycol methyl ether, dipropylene glycol propyl ether, dipropylene glycol butyl ether, dipropylene glycol pentyl ether, dipropylene glycol hexyl ether, dipropylene glycol benzyl ether, mono-methyl polyethylene glycol 5,000 propionic acid, mono-methyl polyethylene glycol 5,000 acetic acid, mono-methyl polyethylene glycol 20,000 2-(succinylamino)ethyl ether, O-(2-carboxyethyl)-O′-methyl-undecaethylene glycol, polyethylene glycol 2,000 monomethyl ether succinate, and the like.

Reaction conditions for the reaction of the carboxylic acid with the glycol ether alcohol to produce compound of Formula F-I, such as temperature, pressure and contact time, may also vary greatly and any suitable combination of such conditions may be employed herein. The reaction temperature can be within a range selected from any one of 25° C. to about 300° C., 50° C. to about 250° C., and 100° C. to about 200° C. The reaction can be carried out under ambient pressure and the contact time may vary from a matter of seconds or minutes to a few hours or greater. The reactants can be added to the reaction mixture or combined in any order. The reaction residence time employed can be within a range selected from any one of about 30 seconds to about 48 hours, about 5 minutes to 36 hours, and about 1 hour to 24 hours.

In an aspect, a single alcohol of Formula F-IV is used in the esterification reaction to produce a single ester of the present disclosure and/or a lubricating oil base stock containing an ester of the present disclosure. In such case, if an acid/anhydride of a single mono-acid is used, a high-purity ester compound of Formula F-I can be obtained and used as a lubricating oil base stock. This is illustrated in Examples 1 and 2 of the present disclosure.

It is also contemplated that multiple alcohols can be used in the esterification reaction. In the case where two different alcohols and the acid/anhydride of a single mono-acid are used in the reaction, the reaction mixture will include two different ester compounds. The ratio between the quantities of the two ester compounds can change as a function of the ratio between the quantities of the two alcohols used. In certain situations, where a mixture of alcohols having similar molecular weights and structures can be procured at a lower cost than a pure alcohol compound, this embodiment can be highly economic to produce a mixture of ester compounds with similar molecular structures, molecular weights, and properties suitable as a lubricating oil base stock product.

In the gamma-branched alcohol of Formula F-IV, R¹ and R² can be independently linear alkyl groups such as: ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-icosyl, n-henicosyl, n-docosyl, n-tricosyl, n-tetracosyl, n-pentacosyl, n-hexacosyl, n-octacosyl, and n-triacontyl. In an aspect, the total number of carbon atoms in linear R¹ and/or R² is an even number. In an aspect, the total number of carbon atoms in linear R¹ and R² combined is from a1 to a2, where a1 and a2 can be, independently, 8, 10, 12, 14, 16, 18, 20, 22, 24, 26, 28, 30, 32, 34, 36, 38, 40, 42, 44, 46, 48, as long as a1<a2. In an aspect, the total number of carbon atoms in linear R¹ and R² combined is within a range selected from any one of 8 to 48, 8 to 40, 8 to 32, 8 to 28, 8 to 26, 8 to 24, 8 to 22, and 8 to 20.

In the gamma-branched alcohol of Formula F-IV, the total number of carbon atoms in linear R¹ and R² combined can be from b1 to b2, where b1 and b2 can be, independently, 8, 10, 12, 14, 16, 18, 20, 22, 24, 26, 28, 30, 32, 34, 36, 38, 40, 42, 44, 46, 48, as long as b1<b2. In an aspect, the total number of carbon atoms in R¹ and R² combined is in a range selected from any one of 8 to 48, 8 to 40, 8 to 32, 8 to 28, 8 to 26, 8 to 24, 8 to 22, and 8 to 20.

In the gamma-branched alcohol of Formula F-IV, the difference in carbon numbers contained R¹ and R² can be two (2). In such case, both R¹ and R² can contain even number of carbon atoms. Thus, one of R¹ and R² may contain 4, 6, 8, 10, 12, 14, 16, 18, 20, 22, 24, 26, or 28 carbon atoms, and the other contains two more carbon atoms. In an aspect, one of R¹ and R² may contain 6, 8, 10, 12, 14, 16, 18, 20, or 22 carbon atoms, and the other contains two more carbon atoms. In an aspect, both R¹ and R² can be linear alkyl groups. A class of such gamma-branched alcohols where both R¹ and R² are linear alkyl groups can be produced according to Scheme I. In Scheme I, an alpha-olefin, having alkyl group R^{g}, is reacted to produce a dimerized to form a branched olefin, which is then reacted by hydroformylation to generate its corresponding aldehyde, which is then reduced (e.g., by hydrogenation) to yield the final gamma-branched alcohol.

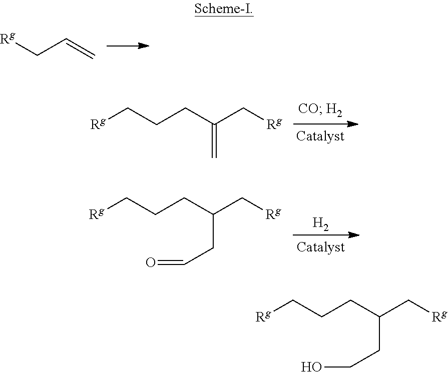

R^{g} can be an alkyl group in an aspect, a linear alkyl group and/or a linear alkyl group having an even number of carbon atoms. Dimerization of the olefin R^{g}—CH₂—CH═CH₂ in the first reaction shown above can be affected in the presence of a catalyst system such as one containing a metallocene compound. Specific examples of Scheme-I are provided in Part A of the Examples of the present disclosure. As can be seen from Scheme-I, where Rg is a linear alkyl group, the final alcohol produced contains two linear alkyl groups (R^{g}—CH₂— and R^{g}—CH₂CH₂CH₂—) connected to the gamma-carbon that differs in terms of number of carbon atoms contained therein by two (2). Many linear alpha-olefins represented by formula R^{g}—CH₂—CH═CH₂ are commercially available, such as 1-butene, 1-pentene, 1-hexene, 1-octene, 1-decene, 1-dodecene, 1-tetradecene, 1-hexadecene, 1-octadecene, and the like, which can be used to fabricate gamma-branched alcohol 3-ethylheptan-1-ol, 3-propyloctan-1-ol, 3-butylnonan-1-ol, 3-hexylundecan-1-ol, 3-octyltridecan-1-ol, 3-decylpentadecan-1-ol, 3-dodecylheptadecan-1-ol, 3-tetradecylnonadecan-1-ol, 3-hexadecylhenicocan-1-ol, and 3-octadecyltricosan-1-ol, respectively.

Examples of gamma-branched alcohols useful in the process of the present disclosure include the following: 3-ethylheptan-1-ol; 3-propyloctan-1-ol, 3-butylnonan-1-ol; 3-hexylundecan-1-ol; 3-octyltridecan-1-ol; 3-decylpentadecan-1-ol; 3-dodecylheptadecan-1-ol; and the like.

The catalyst used in the esterification reaction can be an acid, including strong acids such as p-toluenesulfonic acid monohydride (PTSA), titanium isopropoxide and sulfuric acid.

In an aspect, the reaction can be carried out in the presence of a solvent. The specific solvent used is not critical as long as it is inert in the reaction. Non-limiting examples of the solvent are the following and mixtures thereof: benzene, toluene, xylenes, ethylbenzene, n-pentane and isomers thereof, n-hexane and isomers thereof, n-heptane and isomers thereof, n-octane and isomers thereof, and cyclohexane and saturated isomers thereof. Examples of solvents are the following and mixtures thereof: toluene, n-hexane and isomers thereof, cyclohexane and saturated isomers thereof, ethylbenzene, and any xylene and mixtures thereof.

The reaction mixture from the esterification reaction may include the intended ester product(s), water, and one or more of unreacted acid/anhydride and alcohol, and byproducts such as ethers, and esters of the acid catalyst. Continuous removal of water from the reaction system can result in higher yield of the ester compounds. Components in the reaction mixture having a boiling point lower than the intended gamma-branched alcohol-derived ester can be removed by vacuum. Purification methods such as solvent extraction, chromatography, distillation, and the use of sorbents can be carried out to remove byproducts from reaction mixture to finally obtain one compound of Formula F-I, or a mixture of multiple compounds of Formula F-I, depending on the reactants used, which can be used as a base stock product, or combined with other, similar compounds to form a base stock product.

### Lubricating Oil Compositions Containing Gamma-Branched Alcohol-Derived Ester General

The gamma-branched alcohol-derived ester base stocks of this disclosure are useful in formulating lubricating oils. The oil composition of the present disclosure summarized above can be a portion or the entirety of a lubricating oil formulation ready to be used in its intended application. Thus, the oil composition can be: (i) a mixture of the gamma-branched alcohol-derived ester base stock and the remainder of the formulation absent the gamma-branched alcohol-derived ester base stock; (ii) a mixture of the gamma-branched alcohol-derived ester base stock with one or more other base stocks contained in the lubricating oil formulation absent the additive components in the lubricating oil formulation; (iii) a mixture of the gamma-branched alcohol-derived ester base stock and all other base stocks contained in the lubricating oil formulation but absent any additive components that may be present in the lubricating oil formulation; (iv) a mixture of the gamma-branched alcohol-derived ester base stock and one or more other base stocks, but not all the other base stocks, contained in the lubricating oil formulation, and at least a portion of the additive components contained in the lubricating oil formulation; and (v) a mixture of the gamma-branched alcohol-derived ester base stock and all additive components contained in the lubricating oil formulation, but no other base stocks contained in the lubricating oil formulation.

Therefore, to make a final lubricating oil formulation as a product, one may add additional components, such as other base stocks, additional quantities of the materials already present in the oil composition, additive components, and the like, to the oil composition.

The gamma-branched alcohol-derived ester base stock can be present in the lubricating oil formulation of this disclosure in an amount from about c1 to c2 wt %, based on the total weight of the oil composition, where c1 and c2 can be, independently, 1, 2, 3, 4, 5, 6, 7, 8, 9, 10, 15, 20, 25, 30, 35, 40, 45, 50, 55, 60, 65, 70, 75, 80, 85, 90, 95, or 99 as long as c1<c2. In an aspect, c1 and c2 can be range limits selected form any one of c1=3 and c2=95, c1=5 and c2=90, c1=5 and c2=80, and c1=5 and c2=50. In general, it is desirable that the lubricating oil formulation contains the gamma-branched alcohol-derived ester base stock as a co-base stock. However, it is also contemplated that the lubricating oil formulation of this disclosure may contain the gamma-branched alcohol-derived ester base stock as a primary base stock, and in an extreme case, the lubricating oil formulation may consist essentially of a gamma-branched alcohol-derived ester base stock and additives.

Owing to the high polarity of the gamma-branched alcohol-derived ester base stocks resulting from the ester group in their molecular structures, the lubricating oil compositions of the present disclosure can have an improved polar additive and sludge solvency and dispersancy compared to other lubricating oil compositions free of ester-type base stocks. In addition, a lubricating oil formulation including a gamma-branched alcohol-derived ester base stock can have improved seal compatibility compared to formulations free of ester-type base stocks.

### Other Base Stocks Useful in the Lubricating Oil

A wide range of lubricating oil base stocks known in the art can be used in conjunction with the gamma-branched alcohol-derived ester base stock in the lubricating oil formulations of the present disclosure, as a primary base stock or a co-base stock. Such other base stocks can be either derived from natural resources or synthetic, including un-refined, refined, or re-refined oils. Un-refined oil base stocks include shale oil obtained directly from retorting operations, petroleum oil obtained directly from primary distillation, and ester oil obtained directly from a natural source (such as plant matters and animal tissues) or directly from a chemical esterification process. Refined oil base stocks are those un-refined base stocks further subjected to one or more purification steps such as solvent extraction, secondary distillation, acid extraction, base extraction, filtration, and percolation to improve the at least one lubricating oil property. Re-refined oil base stocks are obtained by processes analogous to refined oils but using an oil that has been previously used as a feed stock.

API Groups I, II, III, IV and V are broad categories of base stocks developed and defined by the American Petroleum Institute (API Publication 1509; www.API.org) to create guidelines for lubricating oil base stocks. Group I base stocks generally have a viscosity index of from about 80 to 120 and contain greater than about 0.03% sulfur and less than about 90% saturates. Group II base stocks generally have a viscosity index of from about 80 to 120, and contain less than or equal to about 0.03% sulfur and greater than or equal to about 90% saturates. Group III base stocks generally have a viscosity index greater than about 120 and contains less than or equal to about 0.03% sulfur and greater than about 90% saturates. Group IV includes polyalphaolefins (PAO). Group V base stocks include base stocks not included in Groups I-IV. Table 1 below summarizes properties of each of these five groups.

TABLE 1
Base Stock Properties

| | Saturates | Sulfur | Viscosity Index |
|---|---|---|---|
| Group I | Higher than | Higher than | At least 80 and |
| | 90 and/or | 0.03% and | at most 120 |
| Group II | Higher than 90 and | At most 0.03% and | At least 80 and |
| | | | at most 120 |
| Group III | At least 90 and | At most 0.03% and | At least 120 |

| | |
|---|---|
| Group IV | PAO products |
| Group V | All other products not included in Groups I, II, III, and IV |

Natural oils include animal oils (e.g. lard), vegetable oils (e.g., castor oil), and mineral oils. Animal and vegetable oils possessing favorable thermal oxidative stability can be used. Of the natural oils, mineral oils are preferred. Mineral oils vary widely as to their crude source, e.g., as to whether they are paraffinic, naphthenic, or mixed paraffinic-naphthenic. Oils derived from coal or shale are also useful in the present disclosure. Natural oils vary also as to the method used for their production and purification, e.g., their distillation range and whether they are straight run or cracked, hydrorefined, or solvent extracted.

Group II and/or Group III base stocks are generally hydroprocessed or hydrocracked base stocks derived from crude oil refining processes.

Synthetic base stocks include polymerized and interpolymerized olefins (e.g., polybutylenes, polypropylenes, propylene isobutylene copolymers, ethylene-olefin copolymers, and ethylene-alphaolefin copolymers).

Synthetic polyalphaolefins (“PAO”) base stocks are placed into Group IV. Advantageous Group IV base stocks are those made from one or more of C6, C8, C10, C12, and C14 linear alpha-olefins (“LAO”s). These base stocks can be commercially available at a wide range of viscosity, such as a KV100 in the range from 1.0 to 1,000 cSt. The PAO base stocks can be made by polymerization of the LAO(s) in the presence of Lewis-acid type catalyst or a metallocene compound-based catalyst system. High quality Group IV PAO commercial base stocks include the SpectraSyn™ and SpectraSyn Elite™ series available from ExxonMobil Chemical Company having an address at 4500 Bayway Drive, Baytown, Tex. 77520, United States.

All other synthetic base stocks, including but not limited to alkyl aromatics and synthetic esters are in Group V.

Additional esters not in the gamma-branched alcohol-derived ester category in a minor amount may be useful in the lubricating oil formulations of this disclosure. Additive solvency and seal compatibility characteristics may be imparted by the use of esters such as the esters of dibasic acids with monoalkanols and the polyol esters of monocarboxylic acids. Esters of the former type include, e.g., the esters of dicarboxylic acids such as phthalic acid, succinic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc. Useful ester-type Group V base stock include the Esterex™ series commercially available from ExxonMobil Chemical Company.

One or more of the following may be used as a base stock in the lubricating oil of the present disclosure as well: (1) one or more Gas-to-Liquids (GTL) materials; and (2) hydrodewaxed, hydroisomerized, solvent dewaxed, or catalytically dewaxed base stocks derived from synthetic wax, natural wax, waxy feeds, slack waxes, gas oils, waxy fuels, hydrocracker bottoms, waxy raffinate, hydrocrackate, thermal crackates, foots oil, and waxy materials derived from coal liquefaction or shale oil. Such waxy feeds can be derived from mineral oils or non-mineral oil processing or can be synthetic (e.g., Fischer-Tropsch feed stocks). Such base stocks can include linear or branched hydrocarbyl compounds of C20 or higher, more preferably C30 or higher.

The lubricating oil formulations of the present disclosure can include one or more Group I, II, III, IV, or V base stocks in addition to the gamma-branched alcohol-derived ester base stock. Preferably, Group I base stocks, if any, are present at a relatively low concentration if a high quality lubricating oil is desired. Group I base stocks may be introduced as a diluent of an additive package at a small quantity. Groups II and III base stocks can be included in the lubricating oil formulations of the present disclosure, but preferably only those with high quality, e.g., those having a VI from 100 to 120. Group IV and V base stocks, preferably those of high quality, are desirably included into the lubricating oil formulations of the present disclosure.

### Lubricating Oil Additives

The lubricating oil formulations of the present disclosure can additionally contain one or more of the commonly used lubricating oil performance additives including but not limited to dispersants, detergents, viscosity modifiers, antiwear additives, corrosion inhibitors, rust inhibitors, metal deactivators, extreme pressure additives, anti-seizure agents, wax modifiers, viscosity modifiers, fluid-loss additives, seal compatibility agents, lubricity agents, anti-staining agents, chromophoric agents, defoamants, demulsifiers, densifiers, wetting agents, gelling agents, tackiness agents, colorants, and others. For a review of many commonly used additives and the quantities used, see: (i) Klamann in Lubricants and Related Products, Verlag Chemie, Deerfield Beach, Fla.; ISBN 0-89573-177-0; (ii) “Lubricant Additives,” M. W. Ranney, published by Noyes Data Corporation of Parkridge, N J (1973); (iii) “Synthetics, Mineral Oils, and Bio-Based Lubricants,” Edited by L. R. Rudnick, CRc Taylor and Francis, 2006, ISBN 1-57444-723-8; (iv) “Lubrication Fundamentals,” J. G. Wills, Marcel Dekker Inc., (New York, 1980); (v) Synthetic Lubricants and High-Performance Functional Fluids, 2nd Ed., Rudnick and Shubkin, Marcel Dekker Inc., (New York, 1999); and (v1) “Polyalphaolefins,” L. R. Rudnick, Chemical Industries (Boca Raton, Fla., United States) (2006), 111 (Synthetics, Mineral Oils, and Bio-Based Lubricants), 3-36. Reference is also made to: (a) U.S. Pat. No. 7,704,930 B2; (b) U.S. Pat. No. 9,458,403 B2, Column 18, line 46 to Column 39, line 68; (c) U.S. Pat. No. 9,422,497 B2, Column 34, line 4 to Column 40, line 55; and (d) U.S. Pat. No. 8,048,833 B2, Column 17, line 48 to Column 27, line 12, the disclosures of which are incorporated herein in their entirety. These additives are commonly delivered with varying amounts of diluent oil that may range from 5 wt % to 50 wt % based on the total weight of the additive package before incorporation into the formulated oil. The additives useful in this disclosure do not have to be soluble in the lubricating oil formulations. Insoluble additives in oil can be dispersed in the lubricating oil formulations of this disclosure.

When lubricating oil formulations contain one or more of the additives discussed above, the additive(s) are blended into the oil composition in an amount sufficient for it to perform its intended function.

It is noted that many of the additives are shipped from the additive manufacturer as a concentrate, containing one or more additives together, with a certain amount of base oil diluents.

Examples of techniques that can be employed to characterize the gamma-branched alcohol-derived ester base stock described above include, but are not limited to, analytical gas chromatography, nuclear magnetic resonance, thermogravimetric analysis (TGA), inductively coupled plasma mass spectrometry, differential scanning calorimetry (DSC), and volatility and viscosity measurements.

The present invention is further illustrated by the following non-limiting examples.

### EXAMPLES

In the following examples, kinematic viscosity at 100° C. (“KV100”) and 40° C. (“KV40”) of fluids were determined pursuant to ASTM standards D-445; viscosity index (“VI”) was determined pursuant to ASTM standard D-2270; and Noack volatility (“NV”) were determined using thermal gravimetric analysis (“TGA”).

### Synthesis of Novel Compounds

By changing the glycol ether component of the gamma-branched alcohol-derived esters in accordance with the present disclosure, the overall polarity of fluids treated with such compounds can be modified. For example, the polarity of a gamma-branched alcohol-derived ester may be modified by varying the R³ glycol ether group of formula (F-I) with any number of synthetic and/or commercially available glycol ethers. For example, glycol ethers such as di(ethylene glycol) monohexyl ether, tri(ethylene glycol) monomethyl ether, tri(propylene glycol) monomethyl ether, tri(ethylene glycol) monoethyl ether, tri(ethylene glycol) monobutyl ether, di(ethylene glycol) monoethyl ether, di(ethylene glycol) monobutyl ether, tri(propylene glycol) monopropyl ether, tri(propylene glycol) monobutyl ether, poly(ethylene glycol) dodecyl ether (Brij 30), and ethylene glycol mono-2-ethylhexyl ether can be used. Glycol ethers, having both ether and alcohol functional groups in the same molecule, represent a versatile class of organic solvents. The Dow Chemical Company manufactures this glycol ether in large quantities. DOW glycol ether products are produced through continuous processes of selectively reacting an alcohol (ethanol, butanol, hexanol) with ethylene oxide. Diethylene glycol monohexyl ether ((C₆H₁₃(OCH₂CH₂)₂OH, Hexyl CARBITOL Solvent) displays a strong hydrocarbon-type solvency.

### Example 1

### Synthesis of 3-octyltridecyl 2-(2-methoxyethoxy)acetate

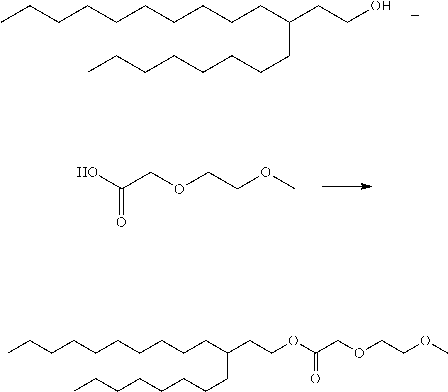

3-octyltridecan-1-ol (7 g, 0.0224 mol, MW 312.58), 2-(2-methoxyethoxy)acetic acid (6.0 g 0.0448 mol, MW 134.13) and p-toluenesulfonic acid monohydride (PTSA) (2.13 g, 0.0112 mol, MW 190.22) were mixed 75 ml toluene in three necked round bottom flask along with a dean-stark apparatus. Then solution was reflux for overnight (18 h). In 18 hours, ˜2-3 ml water was collected in the trap. Toluene was removed by simple distillation at 50° C. The extracted product in methylene chloride washed with water (1×100 ml) and 5% NaOH (1×100 ml). Evaporated the methylene chloride and fallowed by flash chromatography with hexane. The hexane layer is removed by roto-vap at 60° C. under vacuum and high boiling components by air bath oven at 190° C. The isolated product was characterized by IR, 1HNMR, 13CNMR. Yields: 6.5 g (68%). 13C NMR (CDCl3): 170.56, 71.92, 70.75, 68.65, 63.46, 58.94, 34.49, 33.53, 32.38, 31.86, 29.98, 29.66, 29.64, 29.62, 29.59, 29.33, 26.47, 22.65, 14.06

### Example 2

### Synthesis of 3-octyltridecyl 2-(2-(2-methoxyethoxy)ethoxy)acetate

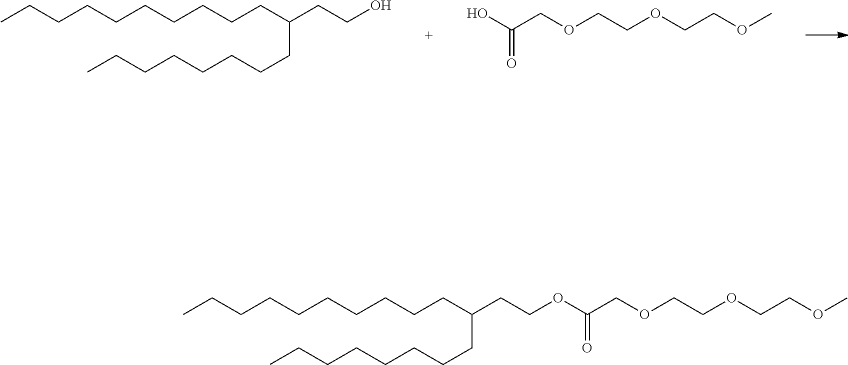 [SMILES: CCCCCCCCCCC(CCO)CCCCCCCC ; COCCOCCOCC(=O)O ; CCCCCCCCCCC(CCCCCCCC)CCOC(=O)COCCOCCOC]

3-octyltridecan-1-ol (7 g, 0.0224 mol, MW 312.58), 2-(2-(2-methoxyethoxy) ethoxy acetic acid (7.98 g 0.0448, MW 178.18) and p-toluenesulfonic acid monohydride (PTSA) (2.13 g, 0.0112 mol, MW 190.22) were mixed 75 ml toluene in three necked round bottom flask along with a dean-stark apparatus. Then solution was reflux for overnight (18 h). In 18 hours, ˜2-3 ml water was collected in the trap. Toluene was removed by simple distillation at 50° C. The extracted product in methylene chloride washed with water (1×100 ml) and 5% NaOH (1×100 ml). Evaporated the methylene chloride and fallowed by flash chromatography with hexane. The hexane layer is removed by roto-vap at 60° C. under vacuum and high boiling components by air bath oven at 190° C. The isolated product was characterized by IR, 1HNMR, 13CNMR. Yields: 6.8 g (64%). 13C NMR (CDCl₃): 170.56, 71.92, 70.86, 70.64, 70.52, 68.69, 63.42, 58.94, 34.52, 33.48, 32.32, 31.89, 29.99, 29.58, 29.33, 26.47, 22.62, 14.09.

### Example 3

### Lube Properties of Base Stocks

The kinematic viscosity (Kv) of the liquid product was measured using ASTM standards D-445 and reported at temperatures of 100° C. (Kv at 100° C.) or 40° C. (Kv at 40° C.). The viscosity index (VI) was measured according to ASTM standard D-2270 using the measured kinematic viscosities for each product. The products were evaluated as synthetic base stocks. The ester fluids were evaluated as synthetic base stocks and results shown in Table 2 below.

TABLE 2

| Example # | MW | Kv₁₀₀ cSt | Kv₄₀ cSt | VI | Noack (TGA) |
|---|---|---|---|---|---|
| 1 | 428 | 3.23 | 12.4 | 131 | 11.6 |
| 2 | 472 | 3.77 | 15.1 | 144 | 9.8 |

Among hydrocarbon fluids metallocene catalyst based low viscosity fluids have superior viscosity-volatility characteristics (i.e. mPAO3.4). These fluids have similar structure with ester and glycol ether segment (polarity, low traction) in one of the arms. It is noteworthy that glycol ether-based fluid (Example 1) has similar molecular weight as mPAO3.4 (MW 428 for ester versus 420 for mPAO3.4) but the viscosity of ester is lower and VI is higher.

### Example 4

### Heat and Thermal Conductivity Properties of Base Stocks

In addition to lubrication applications, base stock fluids and circulating fluids can transfer heat from high temperature zones. In lubricated systems, examples of heat sources include heat generated by combustion processes, heat resulting from friction within a lubricated contact, heat created by energy sources, and heat used in manufacturing processes (e.g., paper and steel making).

In some cases, specialized fluids are used for the sole purpose of removing heat from high temperature zones. Examples include coolants used in internal combustion engine applications, and transformer oils used to cool electrical distribution equipment. Formulations containing gamma-branched alcohol-derived esters in accordance with the present disclosure can also meet the requirements for cooling systems to cool battery and power generation systems in electric and hybrid vehicles to dissipate and distribute heat.

Base stock fluids in accordance with the present disclosure formulated as lubricating and/or cooling fluids can remove heat with combinations of conductivity and convection mechanisms. The heat removed can be a function of fluid properties, such as: heat capacity and thermal conductivity; system design, such as selection of materials that determine the heat flow across fluid/surface interfaces; and operational factors, such as fluid flow rate and temperature difference between fluid and the high temperature zone requiring cooling.

In this example the specific heat capacity of the samples was measured and evaluated against a comparative commercial base stock formulation. Results are shown in Table 3.

TABLE 3
Specific Heat Capacity (J/g/° C.) of Base Stocks

| Temp (° C.) | Example 1 | Example 2 | Comparative 1 |
|---|---|---|---|
| 20 | 2.01 | 2.02 | 1.46 |
| 40 | 2.06 | 2.06 | 1.52 |
| 60 | 2.11 | 2.12 | 1.57 |

1. A compound of structural Formula F-I: 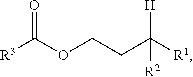
wherein R¹ and R² are independently each a C2 to C30 linear or branched alkyl group; and R³ is a glycol ether or polyglycol ether.

2. The compound of claim 1, wherein R¹ and R² are independently each a linear alkyl group.

3. The compound of claim 1, wherein R¹ and R² are independently each a C2-C24 alkyl group.

4. The compound of claim 1, wherein R¹ and R², taken together, comprise from 8 to 48 carbon atoms.

5. The compound of claim 1, wherein the difference in total number of carbon atoms contained in R¹ and R² is two (2).

6. The compound of claim 4, wherein R² contains more carbon atoms than R¹, and R¹ contains 2, 4, 6, 8, 10, 12, 14, 16, 18, 20, 22, 24, 26, or 28 carbon atoms in total.

7. The compound of claim 1, wherein R³ is a glycol ether having a structure corresponding to the following formula: 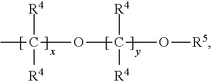
where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), x is a value from about 0 to about 10, and y is a value from about 1 to about 10.

8. The compound of claim 7, wherein R³ comprises 2 to 24 carbon atoms in total.

9. The compound of claim 1, wherein R³ is a polyglycol ether having a structure corresponding to the following formula: 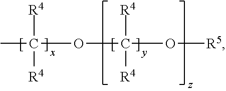
where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30) x is a value from about 0 to about 10, y is a value from about 1 to about 10, and z is a value from about 0 to about 100.

10. The compound of claim 1, wherein the compound is one or more selected from a group consisting of 3-octyltridecyl 2-(2-methoxyethoxy)acetate, 3-octyltridecyl 2-(2-methoxypropoxy)acetate, 3-octyltridecyl 2-(2-(2-methoxyethoxy)ethoxy)acetate, 3-octyltridecyl 2-(2-(2-methoxypropoxy)propoxy)acetate, 3-octyltridecyl 2-(2-(2-methoxypropoxy)ethoxy)acetate, 3-octyltridecyl 2-(2-(2-methoxyethoxy)propoxy)acetate, 3-decyltridecyl 2-(2-methoxyethoxy)acetate, 3-decyltridecyl 2-(2-methoxypropoxy)acetate, 3-decyltridecyl 2-(2-(2-methoxyethoxy)ethoxy)acetate, 3-decyltridecyl 2-(2-(2-methoxypropoxy)propoxy)acetate, 3-decyltridecyl 2-(2-(2-methoxypropoxy)ethoxy)acetate, and 3-decyltridecyl 2-(2-(2-methoxyethoxy)propoxy)acetate.

11. The compound of claim 1, wherein the compound is 3-octyltridecyl 2-(2-methoxyethoxy)acetate or 3-octyltridecyl 2-(2-(2-methoxyethoxy)ethoxy)acetate.

12. A lubricating oil composition comprising a compound of claim 1.

13. The lubricating oil composition of claim 12, wherein the lubricating oil composition is formulated as a coolant for an electric vehicle.

14. The lubricating oil composition of claim 12, which consists essentially of one or more compounds of Formula F-I.

15. The lubricating oil composition of claim 12, having a kinematic viscosity at 100° C. as determined pursuant to ASTM D445 in the range from 1 to 140 cSt.

16. The lubricating oil composition of claim 12, which is a lubricating oil formulation comprising a compound of structural Formula F-I: 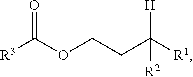
wherein R¹ and R² are independently each a C2 to C30 linear or branched alkyl group; and R³ is a glycol ether or polyglycol ether, as a first base stock.

17. The lubricating oil formulation of claim 16, wherein the concentration of the first base stock, based on the total weight of the lubricant formulation, is in the range from 5 to 95 wt %.

18. The lubricating oil composition of claim 17, further comprising an additive and a second base stock selected from Group I, II, III, IV, and V base stocks.

19. A method for making a compound of Formula F-I or a lubricating oil base stock comprising a compound of Formula F-I: 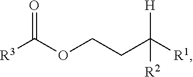
wherein R¹ and R² are independently each a C2 to C30 linear or branched alkyl group and R³ is glycol ether or polyglycol ether; the method comprising the steps of:
reacting an acid of Formula F-II: 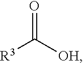
or an anhydride thereof of Formula F-III: 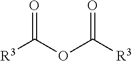
with an alcohol of Formula F-IV in the presence of an acid catalyst to obtain a reaction mixture, 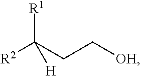
and
obtaining the compound or the lubricating oil base stock from the reaction mixture.

20. The method of claim 19, wherein R and R² are independently each a C2 to C24 linear alkyl group.

21. The method of claim 19, wherein R¹ and R², taken together, comprise from 8 to 48 carbon atoms.

22. The method of claim 19, wherein R³ is a glycol ether having a structure corresponding to the following formula: 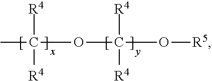
where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30), x is a value from about 0 to about 10, and y is a value from about 1 to about 10.

23. The method of claim 19, wherein R³ is a polyglycol ether having a structure corresponding to the following formula: 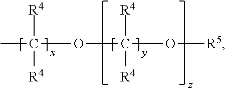
where each R⁴ is the same or different and is hydrogen or a substituted or unsubstituted alkyl group (C1-C30), alkenyl group (C1-C30), alkoxy group (C1-C30), aryl group (C4-C30), or arylalkyl group (C5-C30), R⁵ is hydrogen or a substituted or unsubstituted alkyl group (C1-C30), alkenyl group (C1-C30), alkoxy group (C1-C30), aryl group (C4-C30), or arylalkyl group (C5-C30), x is a value from about 0 to about 10, y is a value from about 1 to about 10, and z is a value from about 0 to about 1).

24. The method of claim 19, wherein the acid catalyst is selected from p-toluenesulfonic acid monohydride (PTSA), titanium isopropoxide and sulfuric acid.

25. The method of claim 19, wherein the compound or the lubricating oil base stock comprises one or more selected from a group consisting of 3-octyltridecyl 2-(2-methoxyethoxy)acetate, 3-octyltridecyl 2-(2-methoxypropoxy)acetate, 3-octyltridecyl 2-(2-(2-methoxyethoxy)ethoxy)acetate, 3-octyltridecyl 2-(2-(2-methoxypropoxy)propoxy)acetate, 3-octyltridecyl 2-(2-(2-methoxypropoxy)ethoxy)acetate, 3-octyltridecyl 2-(2-(2-methoxyethoxy)propoxy)acetate, 3-decyltridecyl 2-(2-methoxyethoxy)acetate, 3-decyltridecyl 2-(2-methoxypropoxy)acetate, 3-decyltridecyl 2-(2-(2-methoxyethoxy)ethoxy)acetate, 3-decyltridecyl 2-(2-(2-methoxypropoxy)propoxy)acetate, 3-decyltridecyl 2-(2-(2-methoxypropoxy)ethoxy)acetate, and 3-decyltridecyl 2-(2-(2-methoxyethoxy)propoxy)acetate.
